(19) United States
(12) Patent Application Publication
(10) Pub. No.: US 2017/0095261 A1
(43) Pub. Date: Apr. 6, 2017

(54) SURGICAL BONE-DRILLING DEVICE, HANDPIECE FOR SUCH A SURGICAL BONE-DRILLING DEVICE, AND METHOD FOR COOLING SUCH A SURGICAL BONE-DRILLING DEVICE

(71) Applicant: Medizinische Universitaet Wien, Wien (AT)

(72) Inventors: Georg Strbac, Wien (AT); Ewald Unger, Wien (AT); Katharina Giannis, Wien (AT)

(21) Appl. No.: 15/311,197

(22) PCT Filed: May 15, 2015
(86) PCT No.: PCT/AT2015/050123

§ 371 Date: Nov. 15, 2016

(30) Foreign Application Priority Data
May 15, 2014 (AT) A 50342/2014

(51) Int. Cl. A61B 17/16; A61C 8/00; A61C 3/02; A61C 1/00; A61C 1/12

(52) U.S. Cl. CPC A61B 17/1644; A61B 17/1626; A61C 1/0069; A61C 1/0092; A61C 1/12; A61C 3/02; A61C 8/0089; A61B 2017/1651; A61B 2017/00199

(57) ABSTRACT

The invention relates to a surgical bone-drilling device (1) comprising a drill (3) connected to a drive (2), a container (4) for a coolant (5), at least one pump (6) for delivering the coolant (5), a first conduit (7) for delivering the coolant (5) from the container (4) into the interior of the drill (3), and a second conduit (8) for delivering the coolant (5) from the container (4) to the outside of the drill (3), and also a handpiece (19), a drill guide (20), and a cooling method for such a bone-drilling device (1). To improve the cooling action, a measuring device (9) is provided for detecting the drilling depth (Δd) of the drill (3), which measuring device (9) is connected to a control device (10) for controlling transport of the coolant (5), such that the quality of the coolant (5) in the first conduit (7) and in the second conduit (8) can be controlled as a function of the drilling depth (Δd) of the drill (3).

The invention relates to a surgical bone drilling device comprising a drill connected to a drive, a container for a coolant, at least one pump for supplying the coolant, a first conduit for supplying the coolant from the container into the interior of the drill and a second conduit for supplying the coolant from the container to the exterior of the drill.

The invention also relates to a contra-angle handpiece for a surgical bone drilling device as defined above, comprising a drill which can be connected to a drive and a first conduit for supplying a coolant into the interior of the drill, and a second conduit for supplying a coolant to the exterior of the drill.

Furthermore, the invention relates to a drill guide for a surgical bone drilling device as defined above, having opening for guiding a drill.

Finally, the present invention also relates to a method for cooling a surgical bone drilling device as defined above, comprising a drill connected to a drive, wherein a coolant is transported by at least one pump from a container via a first conduit into the interior of the drill and via a second conduit onto the exterior of the drill.

The present invention is primarily directed towards dental surgery, although it is also applicable to orthopaedic and neurosurgery. In order to prevent the bone tissue from overheating during surgical drilling procedures into bones, cooling is normally undertaken with the aid of a cooling fluid. In this regard, a distinction is usually made between an internal cooling method, in which the drill is cooled with the aid of a cooling fluid, and an external cooling method, wherein a cooling fluid is supplied to the exterior of the drill in order to dissipate the heat. Current systems usually only allow one of these cooling methods to be selected, and the quantity of coolant is set at a constant value during the drilling procedure.

EP 1 674 046 A1 describes a device for releasable attachment to a handpiece which comprises a conduit for supplying a coolant into the interior of the drill and a conduit for supplying the coolant to the exterior of the drill. There is no description of controlling or adjusting the coolant flowing through the conduits.

WO 2013/173334 A2 discloses a laser device for use in dentistry, in which a fluid is sprayed to cool the operation site; its composition is adjusted as a function of the operative procedure.

WO 2008/055611 A1 describes a dental drill the cooling of which is automatically controlled as soon as contact is made with the operation site. However, in this case it is only possible to switch the cooling on and off.

EP 0 774 238 B1 describes a drill guide for the placement of dental implants which is fitted with coolant channels to guide a cooling fluid. Adjustment of the cooling is not described in that document.

EP 1 639 959 B1 describes a dental treatment device with variable cooling, wherein the quantity of the coolant which is supplied is dependent on the removal capacity of the tool. However, that case concerns the removal of tooth material rather than bone preparations, for which overheating of the bone tissue is far more critical.

Investigations with dental surgical drill systems have shown that temperatures from as low as 47° C. can lead to bone necroses and finally to mechanical damage of the bone to be treated. In known bone drill systems, only 2% of their energy is transformed into kinetic energy, whereas 98% is transformed into heat, and so cooling in order to avoid critical temperatures in the bones is vital. In deep bone preparations, it has been observed that the bone temperature also increases even after the drilling procedure has been completed, and thus can lead to bone necroses (see Strbac G. D., Giannis K., Unger E., Mittlböck M., Vasak C, Watzek G. and Zechner W.: Drilling- and Withdrawing-Related Thermal Changes during Implant Site Osteotomies. Clin. Impl. Dent. and Related Res. 2013 10.1111/cid.12091).

The object of the present invention thus lies in providing a surgical bone drilling device, a handpiece and a drill guide for a surgical bone drilling device of this type, and a method for cooling a surgical bone drilling device of this type, by means of which overheating of the bone to be prepared can be effectively prevented, and thus a bone necrosis and the resulting mechanical problems can be avoided. Disadvantages of known bone drilling devices and methods should be avoided or at least diminished.

The object of the invention is achieved on the one hand by means of the surgical bone drilling device defined above, wherein a measuring device for recording the drilling depth of the drill is provided, the measuring device being connected to a control device for controlling transport of the coolant, so that the quantity of coolant in the first conduit and in the second conduit can be adjusted as a function of the drilling depth of the drill. The present bone drilling device is thus characterized in that the effect of cooling is controlled as a function of the drilling depth, whereby the quantity of coolant is adjusted as a function of the drilling depth. Because the bone drilling device comprises an internal cooling via a first conduit for supplying the coolant from the coolant container into the interior of the drill and an external cooling via a second conduit for supplying the coolant from the container to the exterior of the drill, the quantity of both cooling methods can be correspondingly controlled and be switched, as a function of the control rules, between an internal cooling, external cooling and a combined internal and external cooling with different quantities of coolant. In this manner, in addition to the quantity of coolant, the type of cooling (internal or external) can be selected, or its intensity can be adjusted, and so overheating of the bone tissue can be effectively prevented. The drilling depth can be recorded using various methods, for example optical, magnetic, mechanical or even acoustic measuring devices, which are disposed on the drill device. Depending on the application, the ideal cooling can be selected as a function of the drilling depth; as an example, for shallow drilling depths, it can be switched to primarily the external cooling method, whereas with deep drilling depths, the internal cooling method will be prevalent. In the case of an optimized control of the quantity of coolant, the coolant consumption can also be kept low and the combination of the cooling means that optimized cooling of the bone becomes possible.

Ideally, the control device is configured for supplying the coolant after the drilling procedure is completed, in particular for a predetermined time and/or in a predetermined quantity following completion of the drilling procedure. As already mentioned above, investigations have shown that the temperature in the bone also rises after completion of the drilling procedure. By proceeding to carry out cooling even after the drilling procedure is complete, then in this manner this rise in temperature can be prevented, and thus destruction of the bone tissue can be avoided. The time and/or quantity of coolant which is also used for cooling after completion of the drilling procedure can be adjusted empirically or using specific temperature measurements.

In accordance with one embodiment of the invention, the first conduit and the second conduit are connected to the container for the coolant via a branching element and a common conduit, whereupon the at least one pump is disposed in the common conduit. This variation has the advantage that only one pump is required to transport the coolant for both cooling methods (internal and external). The disadvantage in this regard, however, is that the ratio between the internal and external cooling cannot be influenced without further constructional measures. However, this may be sufficient for certain non-critical applications.

In this regard, a control valve may be disposed in the first conduit and in the second conduit respectively, the control valves being connected to the control device. By appropriately controlling the control valves via the control device, in this manner, switching between internal and external cooling can be carried out and by varying the shut-off timings for the valves or changing the opening characteristic of the valves, the quantity of the coolant can also be adjusted both in the first and in the second coolant conduit as a function of the drilling depth of the drill.

As an alternative to the embodiment described above, in addition, both the first conduit and the second conduit may be connected to the container for the coolant, wherein a pump is disposed in each conduit. This variation allows for individual adjustment of the quantity of coolant in the first conduit for the internal cooling, and also in the second conduit for the external cooling, but this requires two pumps to transport the coolant.

When the control device is connected to an input unit for inputting parameters, the cooling can be further adapted to specific parameters of the drilling procedure. The input unit may be configured in a variety of manners, for example as a barcode reader or keyboard. As an example, the type of drilling being employed, the diameter of the drill being employed or details regarding the bone to be prepared may be input via the appropriately designed input unit, and in this manner, the cooling method can be even better adapted to the respective situation.

Advantageously, the control device is connected to a database. In a database of this type, characteristic values for controlling the cooling which are appropriate to the parameters of the drilling procedure can be stored in a database of this type and be accessed by the control device. The database can be seen as a knowledge database and contains a tabular arrangement of empirical values such as optimal cooling of the drilling method under specific conditions and using specific drills.

The first conduit for supplying the coolant from the container into the interior of the drill and the second conduit for supplying the coolant from the container to the exterior of the drill may be contained in a handpiece as employed in dentistry. In this manner, a handpiece which is employed in dentistry in conventional dental treatment units can be used with the control of cooling in accordance with the invention.

The second conduit for supplying the coolant from the container to the exterior of the drill may also be contained in a drill guide with which the bone drilling procedure is supported. When using a drill guide, which is usual in dental surgery, but also could be used in orthopaedics, the second conduit for supplying the coolant from the coolant container to the exterior of the drill is disposed in this drill guide, while the internal cooling disposed in the drill or the drilling device.

When a temperature measuring device which is connected to the control device is provided for measuring the temperature at the drilling site, an optimized control of cooling can be undertaken as a function of the actual temperature at the drilling site. The temperature measurement may be arranged using appropriate miniaturized sensors in the interior of the drill, or at a corresponding appropriate location, for example also in a drill guide.

In accordance with a further feature of the invention, a device for recording the quantity of coolant in the container is disposed in the container for the coolant. By means of a feature of this type, the physician will be warned in good time before the coolant container becomes empty, so that the drilling procedure can be stopped before there is a risk of overheating of the bone.

Similarly, a device for measuring the quantity of coolant flowing through the conduits may be provided. By means of measuring devices of this type, a malfunction, for example blockage of the conduits, can be detected and the surgeon or dentist can be warned.

By means of a display device which is connected to the measuring device to record the drilling depth of the drill, an optical or acoustic indication of the drilling depth can be provided to the clinician, whereupon he can draw specific conclusions or adjust the drilling procedure after reaching the desired drilling depth.

The first conduit for supplying the coolant from the container into the interior of the drill may be fed back to the container in order to form a closed circuit. In this manner, the consumption of coolant is reduced and the preparation area can be flooded with less coolant. On the other hand, with a closed system, the heat which is transported in the coolant must be dissipated appropriately with the aid of suitable heat exchangers.

The object of the invention is also achieved by means of a handpiece for a surgical bone drilling device, wherein a measuring device for recording the drilling depth of the drill is provided, the measuring device being capable of being connected to a control device for controlling conveying of the coolant, so that the quantity of the coolant supplied through the first conduit and the second conduit can be controlled as a function of the drilling depth of the drill. The above description of the surgical bone drilling device should be referred to with respect to the resulting advantages. The measuring device for recording the drilling depth of the drill is contained in appropriately miniaturized manner in the handpiece and delivers the data via cable or wirelessly to the control device which is connected to the pumps for supplying the coolant. The measurement of the drilling depth may be carried out optically, for example by laser triangulation, mechanically, acoustically by ultrasound, or via the oscillation of the drill and analysis of the resonance behaviour. Providing that appropriate interfaces are retained, a handpiece of this type may be used in conventional dental surgical devices.

Advantageously, a temperature measuring device which can be connected to the control device is provided to measure the temperature at the drilling site. As mentioned above already, recording the actual temperature at the drilling site means that the control procedure can be engaged in an even more proactive manner. The temperature measurement can readily be carried out using appropriate miniaturized temperature sensors.

Furthermore, the object of the invention is also achieved by means of a drill guide for a surgical bone drilling device, wherein a conduit for supplying a coolant into the region of the drilling site is connected to each opening, wherein transport of the coolant in the conduits can be adjusted as a function of the drilling depth of the drill. A drill guide configured in this manner means that optimized adjustment and selection of the suitable cooling method (internal and/or external) and the appropriate quantity of coolant can be obtained.

Finally, the object of the invention is also achieved by means of a method for cooling a surgical bone drilling device, wherein the drilling depth of the drill is measured and the quantity of coolant in the first conduit and in the second conduit is controlled as a function of the measured drilling depth of the drill. The description above relating to the surgical bone drilling device should be referred to in respect of the resulting advantages.

Optionally, parameters, for example the drill employed, can be input via an input unit and the quantity of coolant can be controlled as a function of these parameters. The input unit may, for example, be formed by a keyboard or a touch screen, but also by a barcode reader or another sensor through which the appropriate parameters, for example the type of the drill used or the like, may be input.

Ideally, even after the drilling procedure has been completed, coolant is transported, in particular for a predetermined time and/or in a predetermined quantity. In this manner, an increase in the bone temperature after completion of the drilling procedure is effectively prevented.

When the temperature at the drilling site is measured and the quantity of coolant is controlled as a function of the measured temperature, even better optimized cooling can be obtained.

The quantity of coolant present in the container for the coolant may be measured and advantageously displayed optically or acoustically.

Similarly, the quantity of the coolant transported through the conduits can be measured and provide valuable information to the physician.

When the quantity of coolant transported through the conduits drops below a predetermined quantity, an alarm is emitted, thus increasing the security against overheating of the drilling site.

Cooled physiological saline solution is preferably used as the coolant. Other types of cooling fluids which do not have a negative effect on the body may also be used.

The invention will now be described in more detail with the aid of the accompanying figures, in which:

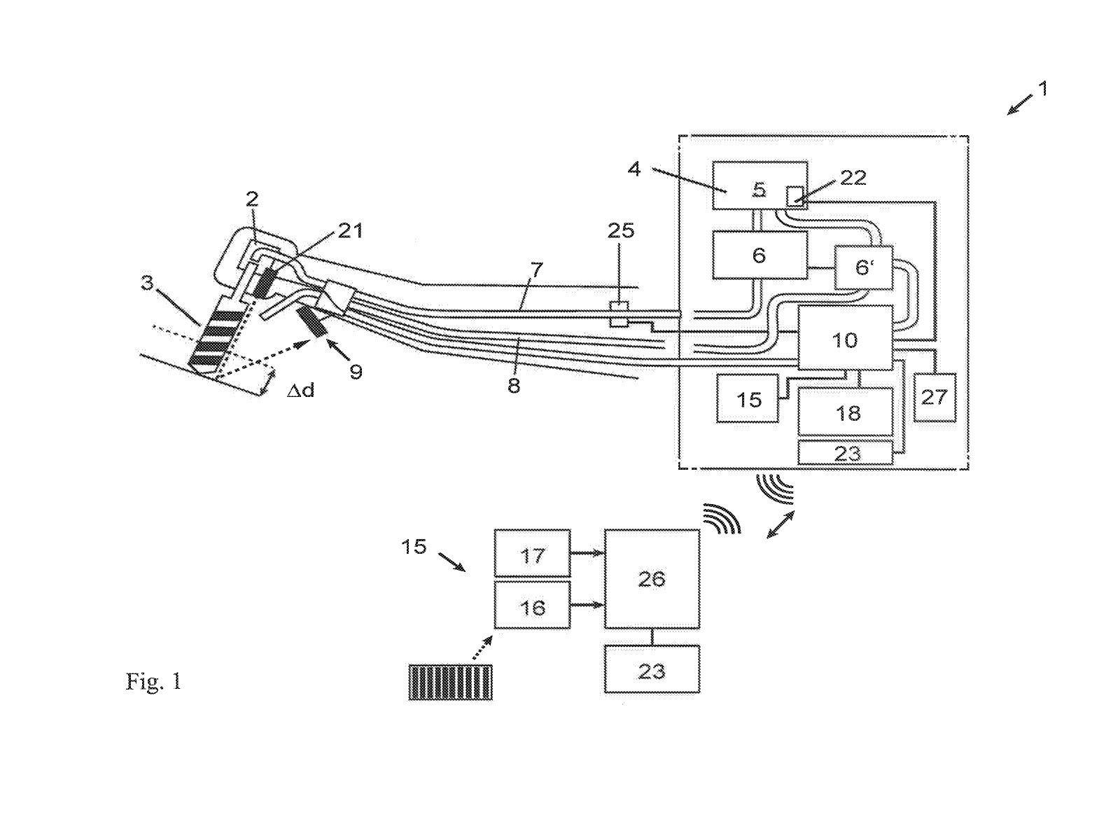

FIG. 1 shows a block diagram of an embodiment of a surgical bone drilling device with combined coolant and control of the cooling as a function of the measured drilling depth of the drill;

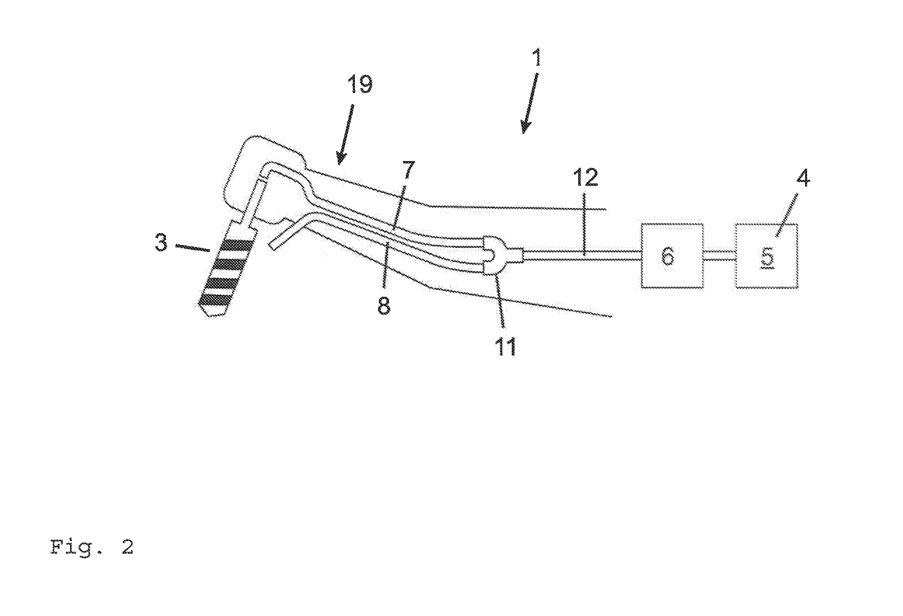

FIG. 2 shows a diagrammatic view of a surgical bone drilling device with combined internal and external cooling with a pump and a Y-piece;

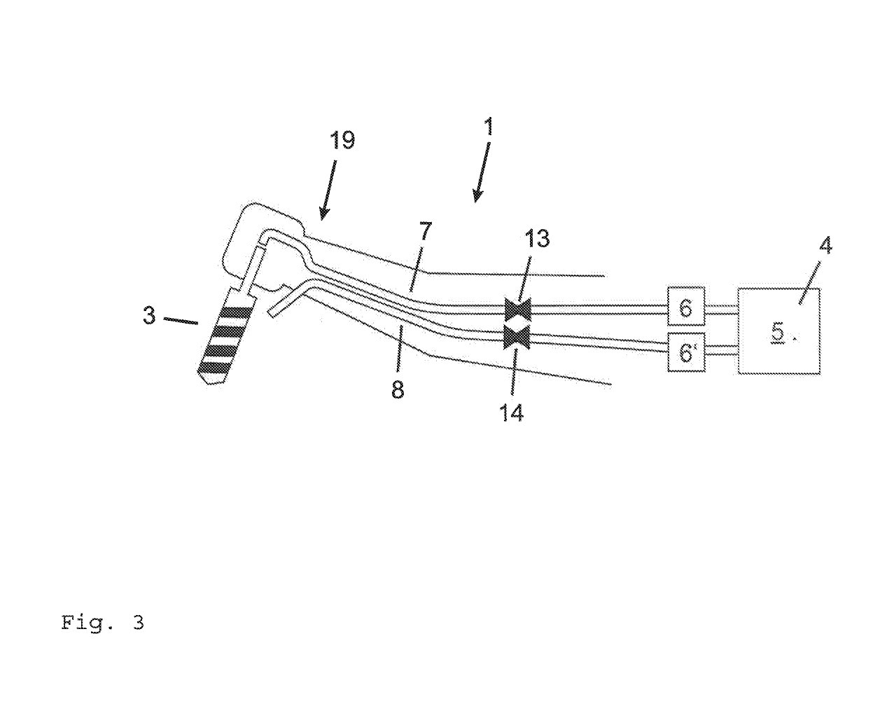

FIG. 3 shows a diagrammatic view of a surgical bone device with combined internal and external cooling with two separate pumps or control valves;

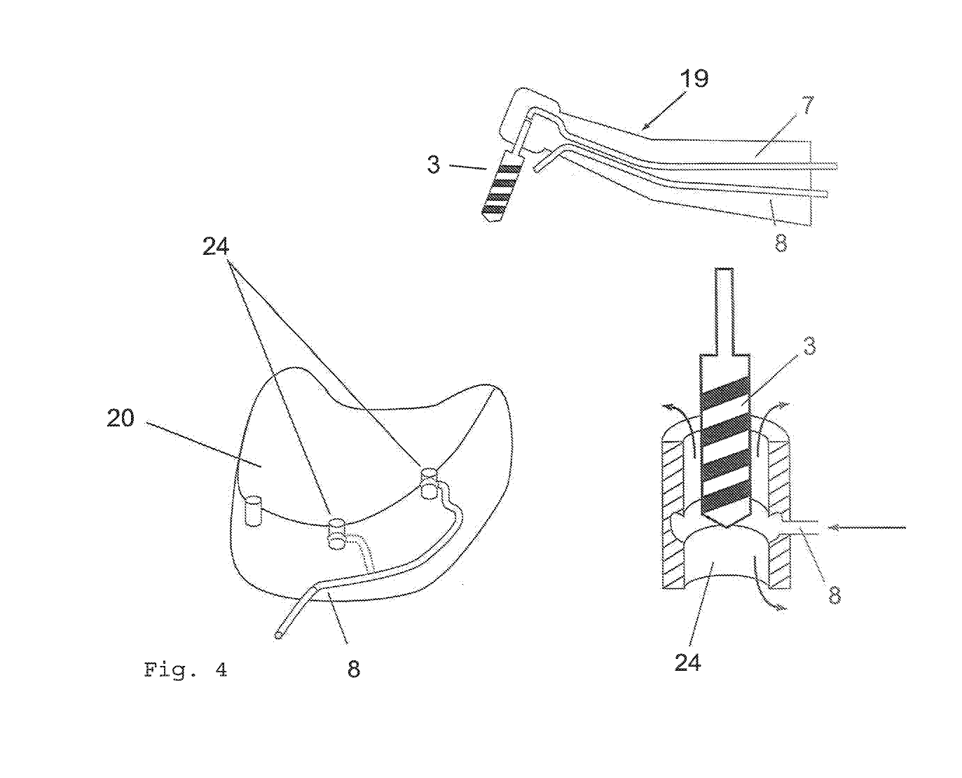

FIGS. 4a-4c show a surgical bone drilling device with internal cooling in the drill head or handpiece and external cooling in a drill guide;

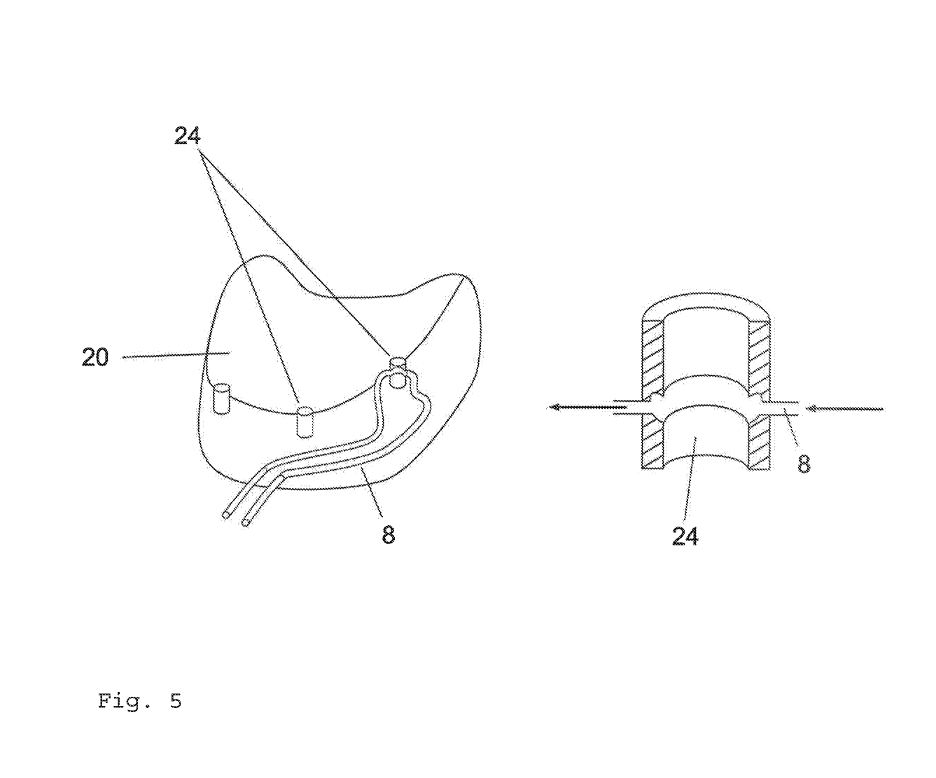

FIGS. 5a-5b show a further embodiment of a surgical bone drilling device with internal cooling in the drill head or handpiece and external cooling in a drill guide;

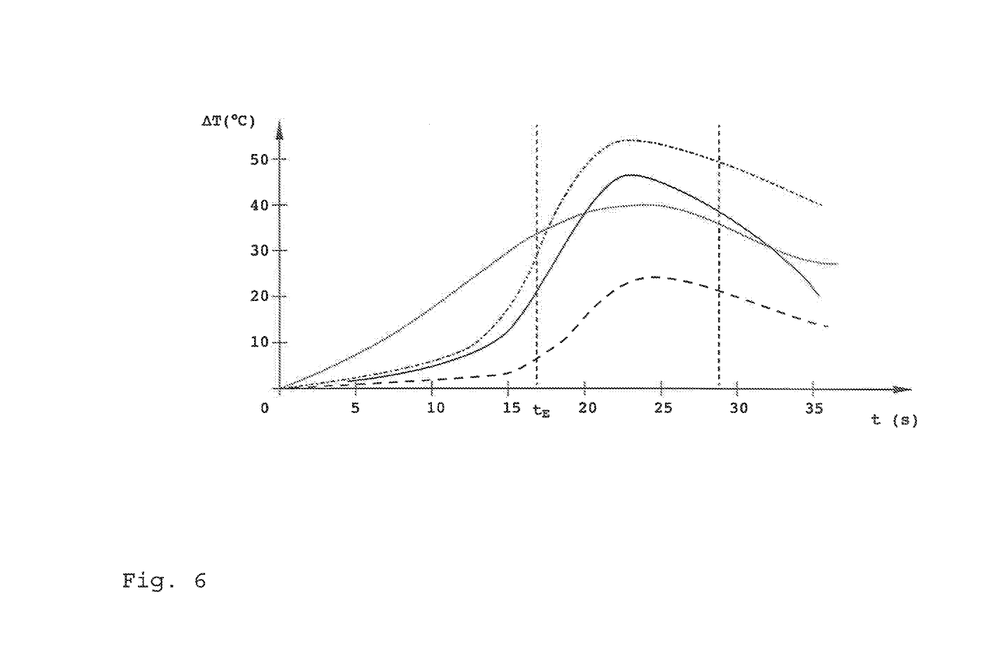

FIG. 6 shows a graph with time to illustrate the temperature profile at the drilling site during and after the drilling procedure in conventional cooling procedures; and

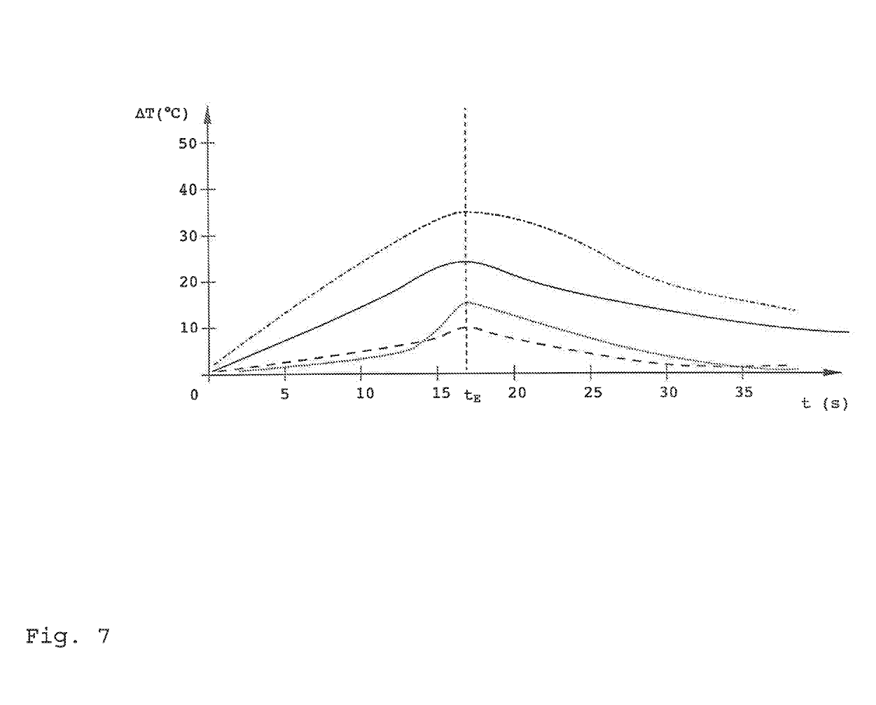

FIG. 7 shows a graph with time to illustrate the temperature profile at the drilling site during and after the drilling procedure in the cooling procedure in accordance with the invention.

FIG. 1 shows a block diagram of an embodiment of a surgical bone drilling device 1 with combined cooling and control of cooling as a function of the measured drilling depth Δd of the drill 3. The surgical bone drilling device 1 comprises a drive 2 for the drill 3 and a container 4 for the coolant 5, for example the physiological saline solution. Coolant 5 is guided from the container 4 into the interior of the drill 3 for internal cooling via a first conduit 7. Coolant 5 is guided from the container 4 to the exterior of the drill 3 for external cooling via a second conduit 8. In the example shown, two pumps 6, 6′ are disposed to supply the coolant 5 in the first conduit 7 and the second conduit 8. In accordance with the invention, a measuring device 9 is provided to record the drilling depth Δd of the drill 3; the measuring device can be configured in various manners, for example by optical, acoustic or mechanical measurement. The measuring device 9 delivers a signal to the control device 10 which is connected to the pumps 6, 6′ so that the quantity of coolant 5 can be controlled as a function of the drilling depth Δd of the drill 3. In this regard, the quantity of coolant 5 in the first conduit 7 and the second conduit 8 is in principle adjusted from 0-100%, whereupon pure internal cooling, pure external cooling and combined internal and external cooling with variable quantities of coolant can be selected. The control device 10 may be connected to an input unit 15, by means of which certain parameters can be input or selected. The input unit 15 may, for example, be formed by a barcode reader 16 or a keyboard 17. The input unit 15 may be disposed either internally or externally; when disposed externally, a wireless connection via a transmitter 26 and a receiver 27 may be provided which is connected to the control device 10, or indeed a hard-wired connection may be provided. Furthermore, the control device 10 may be connected to a database 18 via which important data for implementing the control method may be obtained. The database 18 may also be disposed remotely and be made accessible via an appropriate network, for example the internet. Depending on the specifications for the control device 10, in particular, conveyance of the coolant 5 after completion of the drilling procedure may be provided for, for example for a given time Δt and/or for a given quantity Δm of the coolant 5. In this manner, local overheating of the bone tissue may also be prevented after completion of the drilling procedure.

A temperature measuring device 21 for measuring the temperature at the drilling site may be provided on the surgical bone drilling device 1. This temperature measuring device 21 is connected to the control device 10, which may be configured in a variety of manners. In the container 4 for the coolant 5, a device 22 may be provided to record the quantity of coolant 5 present in the container 4, so that the user can be informed in good time how much coolant 5 remains in the container 4. It is possible to measure the quantity of coolant 5 flowing through the conduits 7, 8 via a device 25 which may be configured in a variety of manners. This device 25 for measuring the quantity of coolant 5 flowing through the conduits 7, 8 is also connected to the control device 10. The user of the surgical bone drilling device 1 can be informed acoustically or optically of various events via a display device 23 which may be connected internally or externally with the control device 10.

FIG. 2 shows a diagrammatic view of a surgical bone drilling device 1 with combined internal and external cooling with a pump 6 and a branching element 11. Here, the first conduit 7 for supplying the coolant 5 from the container 4 into the interior of the drill 3 and the second conduit 8 for conveying the coolant 5 from the container 4 to the exterior of the drill 3 are brought together via the branching element 11 into a common conduit 12 in which a common pump for supplying the coolant 5 is disposed. This variation has the advantage that only one pump 6 is required, however an independent control of the quantity of coolant 5 for the internal cooling and the external cooling or the first conduit. 7 or the second conduit 8 is not possible.

FIG. 3 shows a diagrammatic view of a surgical bone drilling device 1 with combined internal and external cooling with two separate pumps 6, 6′ or control valves 13, 14. In this variational embodiment, although two pumps 6, 6′ or two control valves 13, 14 are required, an independent control of the quantity of coolant 5 for the internal and external cooling can be carried out.

FIGS. 4a-4c show a surgical bone drilling device 1 with internal cooling in the drilling head or handpiece 19 and external cooling in a drill guide 20. The outlined handpiece 19 contains both a first conduit 7 for the internal cooling inside the drill 3, and also a second conduit 8 for the external cooling at the exterior of the drill 3. The drill guide 20 contains the second conduit 8 for supplying the coolant 5 from the container 4 to the openings 24 in the drill guide 20. The coolant 5 reaches the exterior of the drill 3 via the opening 24.

FIGS. 5a and 5b show a variation of the surgical bone drilling device 1 of FIGS. 4a-4c, wherein the external cooling is provided through two respective conduits 8 to the openings for accommodating the drill 3, whereupon a quasi-sealed system is produced and the cooling medium can be practically guided in a closed circuit (naturally a portion of the coolant 5 is lost via the exterior of the drill 3).

Finally, FIG. 6 shows a graph with time to illustrate the temperature profiles at the drilling site during and after the drilling procedure at different drilling depths. The diagram shown shows the temperature profile during (t≦t_{E}) and after (t>t_{E}) a drilling procedure in a jawbone with conventional cooling. In the conventional drilling procedure, it can be seen that after completion of the drilling procedure at time t_{E}, the temperature in the bone climbs further and in the event of exceeding a critical level, bone necrosis can occur. By means of the inventive method for cooling as a function of the drilling depth Δd of the drill 3 of the bone drilling device 1, a temperature rise of this type after completion of the drilling procedure at time t_{E} is effectively prevented, since cooling is continued for an appropriate time Δt after completion of the drilling procedure. Instead of a fixed time Δt, a specific quantity Δm of coolant 5 may also be consumed after completion of the drilling procedure before cooling is finally shut off. The various graphs in FIGS. 6 and 7 were recorded for different drilling depths.

The present invention can be used to provide optimized cooling of surgical bone drilling devices and is of assistance in preventing bone necroses due to exceeding critical temperatures within the bone tissue.

1. A surgical bone drilling device comprising:
a drill connected to a drive,
a container for a coolant,
at least one pump for supplying the coolant,
a first conduit for supplying the coolant from the container into the interior of the drill, and
a second conduit for supplying the coolant from the container to the exterior of the drill, wherein
a measuring device is provided for recording a drilling depth (Δd) of the drill, the measuring device being connected to a control device for controlling transport of the coolant, so that a quantity of coolant in the first conduit and in the second conduit can be adjusted as a function of the drilling depth (Δd) of the drill.

2. The surgical bone drilling device as claimed in claim 1, wherein the control device is configured to transport the coolant after completion of the drilling procedure for a predetermined time (Δt) and/or in a predetermined quantity (Δm) following completion of the drilling procedure.

3. The surgical bone drilling device as claimed in claim 1, wherein the first conduit and the second conduit are connected to the container for the coolant via a branching element and a common conduit, wherein the at least one pump is disposed in the common conduit.

4. The surgical bone drilling device as claimed in claim 1, wherein a control valve is disposed in the first conduit and in the second conduit respectively, the control valves being connected to the control device.

5. The surgical bone drilling device as claimed in claim 1, wherein the first conduit and the second conduit are connected to the container for the coolant, wherein a pump is disposed in each conduit.

6. The surgical bone drilling device as claimed in claim 1, wherein the control device is connected to an input unit for inputting parameters.

7. The surgical bone drilling device as claimed in claim 1, wherein the control device is connected to a database.

8. The surgical bone drilling device as claimed in claim 1, wherein the first conduit for supplying the coolant from the container into the interior of the drill and the second conduit for supplying the coolant from the container to the exterior of the drill are contained in a handpiece.

9. The surgical bone drilling device as claimed in claim 1, wherein the second conduit for supplying the coolant from the container to the exterior of the drill is contained in a drill guide.

10. The surgical bone drilling device as claimed in claim 1, wherein a temperature measuring device connected to the control device is provided in order to measure the temperature (T) at the drilling site.

11. The surgical bone drilling device as claimed in claim 1, wherein a device adapted to record quantity of coolant in the container is disposed in the container for the coolant.

12. The surgical bone drilling device as claimed in claim 1, wherein a device is provided that is adapted to measure a quantity of coolant flowing through the first and second conduit.

13. The surgical bone drilling device as claimed in claim 1, wherein a display device is provided which is connected to the measuring device which is adapted to record the drilling depth (Δd) of the drill.

14. The surgical bone drilling device as claimed in claim 1, wherein the first conduit for supplying the coolant from the container into the interior of the drill is fed back to the container in order to form a closed circuit.

15. A handpiece for a surgical bone drilling device, comprising a drill which is adapted to be connected to a drive and a first conduit for supplying a coolant into the interior of the drill, and a second conduit for supplying a coolant to the exterior of the drill, wherein a measuring device is provided and is adapted to record a drilling depth (Δd) of the drill, the measuring device being capable of being connected to a control device for controlling transport of the coolant, so that the quantity of the coolant supplied through the first conduit and the second conduit can be controlled as a function of the drilling depth (Δd) of the drill.

16. The handpiece as claimed in claim 15, wherein a temperature measuring device is provided at a drilling site to measure the temperature (T).

17. A drill guide for a surgical bone drilling device comprising:
openings for guiding a drill, wherein a conduit for supplying a coolant into the region of the drilling site is connected to each opening (24), wherein transport of the coolant can be controlled in the conduits as a function of a drilling depth (Δd) of the drill.

18. A method for cooling a surgical bone drilling device comprising a drill connected to a drive, wherein a coolant is transported by at least one pump from a container via a first conduit into the interior of the drill and via a second conduit onto the exterior of the drill, wherein a drilling depth (Δd) of the drill is measured and a quantity of coolant in the first conduit and in the second conduit is controlled as a function of the measured drilling depth (Δd) of the drill.

19. The method as claimed in claim 18, wherein parameters, for example the drill employed, are input via an input unit and the quantity of coolant is controlled as a function of these parameters.

20. The method as claimed in claim 18, wherein even after drilling has been completed, the coolant is transported, in particular for a predetermined time (Δt) and/or in a predetermined quantity (Δm) of the coolant.

21. The method as claimed in claim 18, wherein a temperature (T) at a drilling site is measured and the quantity of coolant is controlled as a function of a measured temperature.

22. The method as claimed in claim 18, wherein the quantity of coolant present in the container for the coolant is measured and advantageously displayed optically or acoustically.

23. The method as claimed in claim 18, wherein the quantity of the coolant transported through the conduits is measured.

24. The method as claimed in claim 23, wherein when the quantity of the coolant transported through the first and second conduit drops below a predetermined quantity, an alarm is emitted.

25. The method as claimed in claim 18, wherein cooled physiological saline solution is used as the coolant.

26. The method as claimed in claim 6, wherein the input unit is a barcode reader or a keyboard.
